(19) United States
(12) Patent Application Publication
(10) Pub. No.: US 2024/0398434 A1
(43) Pub. Date: Dec. 5, 2024

(54) SURGICAL DEVICES, SYSTEMS, AND METHODS FOR DETERMINING WEAR

(71) Applicant: Medtronic PS Medical, Inc., Goleta, CA (US)

(72) Inventors: Michael Vu, Grand Prairie, TX (US); Jennifer M. Cao, Mckinney, TX (US); Haoran Li, Flower Mound, TX (US); Aayush Malla, Fort Worth, TX (US); Andrew J. Wald, Fort Worth, TX (US); Saideep Nakka, Northlake, TX (US); Mark W. Garwood, Fort Worth, TX (US); Milton F. Barnes, Grand Prairie, TX (US); Sophie A. Pervere, Dallas, TX (US); John W. Kulas, Euless, TX (US)

(21) Appl. No.: 18/673,448

(22) Filed: May 24, 2024

Related U.S. Application Data
(60) Provisional application No. 63/469,643, filed on May 30, 2023.

(51) Int. Cl. A61B 17/32

(52) U.S. Cl. CPC A61B 17/32

(57) ABSTRACT

A surgical device includes a tool and a motor configured to drive movement of the tool. The tool is supported on a shaft assembly and the shaft assembly and the motor include a plurality of components that cooperate to support or drive the tool. One or more indicators are disposed proximate one or more of the plurality of components and are configured to provide feedback indicative of one or more properties of one of the plurality of components either prior to, during or after use of the surgical device. The plurality of components may include at least one of gears, shafts and bearings, and the indicators may include any one of: thermochromic indicators, thermally-activated elements, thermal fuses, thermocouples, and thermistors.

### CROSS-REFERENCE TO RELATED APPLICATION

This application claims the benefit of and priority to U.S. Provisional Patent Application No. 63/469,643 filed May 30, 2023, the entire disclosure of which is incorporated by reference herein.

### FIELD

The present disclosure relates to surgical devices, systems, and methods and, more particularly, to surgical devices, systems, and methods for determining wear or prolonged use of a surgical device to prevent or avoid failure.

### BACKGROUND

Powered surgical cutting devices and systems are utilized in a wide variety of surgical procedures to perform various different surgical cutting functions including, for example, drilling, tapping, resection, dissection, debridement, shaving, sawing, pulverizing, and/or shaping of anatomical tissue including bone.

Many of such powered surgical cutting devices and systems are precisely designed to ensure the devices function safely and effectively. However, regardless of the precision of design, all powered surgical devices and systems have components that tend to wear over prolonged use or in cases of excessive use increasing the risk of device failure. Providing information to the surgeon regarding the overall state of the various components of the surgical device before, during, and/or after surgery would be of value and allow a surgeon to determine whether to continue or discontinue using a particular surgical device.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. To the extent consistent, any of the aspects described herein may be used in conjunction with any or all of the other aspects described herein.

Provided in accordance with aspects of the present disclosure is a surgical device that includes a tool and a motor configured to drive movement of the tool. The tool is supported on a shaft assembly, and the shaft assembly and the motor include a plurality of components that cooperate to support or drive the tool. One or more indicators is disposed proximate one or more of the plurality of components and is configured to provide feedback indicative of one or more properties of one of the plurality of components either prior to, during or after use of the surgical device. The plurality of components includes at least one of gears, shafts and/or bearings and the one or more indicators may include any one of: thermochromic indicators, thermally-activated elements, thermal fuses, thermocouples, or thermistors.

In aspects according to the present disclosure, the one or more indicators is a thermochromic indicator visibly disposed on an outer surface of the surgical device proximate one or more bearings configured to support the tool, the thermochromic indicator having a wide temperature gradient range to visibly indicate varying states of condition of the one or more bearings of the surgical device.

In aspects according to the present disclosure, the one or more indicators is a thermally-activated element operably associated with one or more bearings configured to support the tool, the thermally-activated element adapted to couple to a power console, e.g., an integrated power console (IPC), to visibly indicate the condition of the one or more bearings of the surgical device.

In aspects according to the present disclosure, the one or more indicators is a thermocouple operably associated with one or more bearings configured to support the tool, the thermocouple adapted to couple to an integrated power console (IPC) to visibly indicate varying states of condition of the one or more bearings of the surgical device.

In aspects according to the present disclosure, the surgical device is adapted to couple to an integrated power console (IPC) configured to monitor feedback from the one or more indicators during use over time to visibly indicate the condition of the surgical device. In other aspects according to the present disclosure, the feedback from the one or more indicators is selected from the group consisting of current, voltage, vibration, and noise.

Provided in accordance with the present disclosure is a method for determining the state of condition of a surgical device and includes driving a motor to move a tool of a surgical device, the tool supported on a shaft assembly, the shaft assembly and the motor including a plurality of components that cooperate to support and/or drive the tool. The method also includes analyzing feedback from one or more indicators disposed proximate one of the plurality of components, the feedback indicative of one or more properties of one (or more) of the plurality of components either prior to, during, or after use of the surgical device. The plurality of components includes at least one of gears, shafts and bearings and the one or more indicators may include any one of: thermochromic indicators, thermally-activated elements, thermal fuses, thermocouples, or thermistors.

In aspects according to the present disclosure, the analyzing feedback from the one or more indicators includes analyzing feedback from an organic thermochromic indicator visibly disposed on an outer surface of the surgical device proximate one or more bearings configured to support the tool, the organic thermochromic indicator having a wide temperature gradient range to visibly indicate varying states of condition of the one or more bearings of the surgical device.

In aspects according to the present disclosure, the analyzing feedback from the one or more indicators includes analyzing feedback from a thermally-activated element operably associated with one or more bearings configured to support the tool, the thermally-activated element adapted to couple to a power console, e.g., an integrated power console (IPC), to visibly indicate the condition of the one or more bearings of the surgical device.

In aspects according to the present disclosure, the analyzing feedback from the one or more indicators includes analyzing feedback from a thermocouple operably associated with one or more bearings configured to support the tool, the thermocouple adapted to couple to an integrated power console (IPC) to visibly indicate varying states of condition of the one or more bearings of the surgical device.

In aspects according to the present disclosure, the method further includes: monitoring feedback from the one or more indicators during use over time to visibly indicate the condition of the surgical device on a power console, e.g., an integrated power console (IPC). In other aspects according to the present disclosure, the feedback from the one or more indicators may be any one of: current, voltage, vibration, or noise.

Provided in accordance with the present disclosure is surgical device including a tool and a motor configured to drive movement of the tool of the surgical device. The tool is supported on a shaft assembly and the shaft assembly and motor include a plurality of components that cooperate to support or drive the tool. A microcontroller includes a sensor operably associated with the shaft assembly, the microcontroller adapted to couple to a power console, e.g., an integrated power console (IPC). The sensor is configured to provide feedback indicative of one or more properties of the surgical device during use which can be compared against a known neural network of patterns or behaviors to anticipate issues, recommend replacement of one or more of the plurality of components, or determine a state of failure of the surgical device.

In aspects according to the present disclosure, the sensor is an accelerometer or a noise sensor.

In aspects according to the present disclosure, the feedback from the sensor may include one or more of: drive current, vibration, or noise.

In aspects according to the present disclosure, the sensor is disposed on a distal end of the shaft assembly.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a surgical system in accordance with the present disclosure including a console and a powered surgical cutting device;

FIGS. 2A and 2B illustrate side views of a powered surgical cutting device for use with the system of FIG. 1 including a thermochromic indicator to monitor the state of one or more components of the surgical device in accordance with one embodiment of the present disclosure;

FIGS. 3A and 3B illustrate side views of a powered surgical cutting device for use with the system of FIG. 1 including a series of thermal fuses operatively associated with one or more components of the surgical device to monitor the state of the one or more components in accordance with another embodiment of the present disclosure;

FIGS. 4A and 4B illustrate side views of a powered surgical cutting device for use with the system of FIG. 1 including a series of thermocouples/thermistors operatively associated with one or more components of the surgical device to monitor the state of the one or more components in accordance with another embodiment of the present disclosure;

FIG. 5A illustrates a side view of a powered surgical cutting device for use with the system of FIG. 1 including a series of contact points on one or more components of the surgical device for monitoring the motor current demand of the one or more components in accordance with another embodiment of the present disclosure;

FIG. 5B is a graph showing a substantially steady motor current demand for one of the components at the respective contact point;

FIG. 6A illustrates a side view of the powered surgical cutting device of FIG. 5A showing excessive or prolonged use of the surgical device resulting in high motor current demand on the one or more components;

FIG. 6B is a graph showing increasing motor current demand on the respective contact point, i.e., component, of FIG. 5B;

FIGS. 7A-7C illustrate various views of a powered surgical cutting device for use with the system of FIG. 1 including a microcontroller having an accelerometer actively monitoring one or more components of the surgical device to determine the state thereof in accordance with another embodiment of the present disclosure;

FIGS. 8A-8C illustrate various views of a powered surgical cutting device for use with the system of FIG. 1 including a microcontroller having a noise sensor that is configured to actively monitor one or more components of the surgical device to determine the state thereof in accordance with another embodiment of the present disclosure;

FIG. 9 illustrates a side view of a powered surgical cutting device for use with the system of FIG. 1 including a vision-based system that actively monitors one or more components of the surgical device to determine the state thereof in accordance with another embodiment of the present disclosure; and

FIGS. 10A-10C illustrate various views of a powered surgical cutting device for use with the system of FIG. 1 including an electrically conductive coating applied to one or more components of the surgical device to determine the state thereof in accordance with another embodiment of the present disclosure.

### DETAILED DESCRIPTION

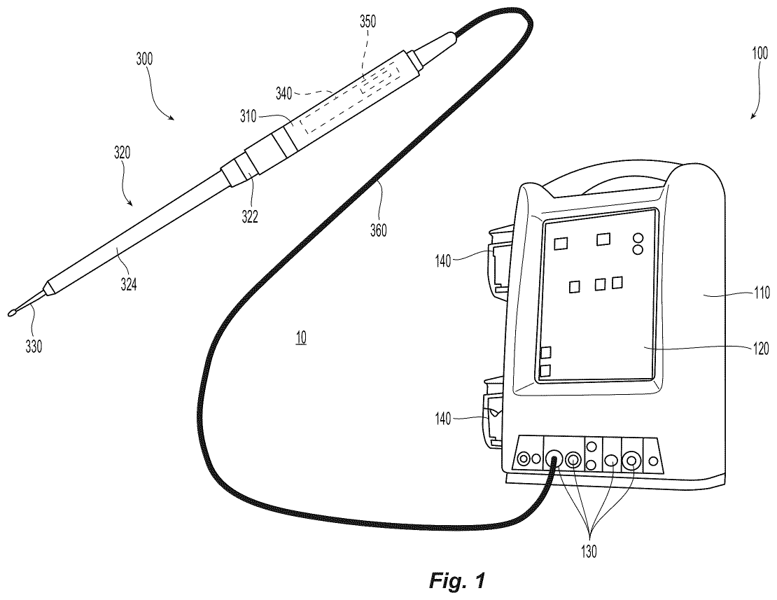

Turning to FIG. 1, a surgical system 10 provided in accordance with the present disclosure includes a console 100 and one or more surgical cutting devices 300. Console 100 may include an outer housing 110 enclosing the internal operable components of console 100, a touch screen graphical user interface (GUI) 120 to receive user input and display information to the user, a plurality of device ports 130, one or more fluid pumps 140, and/or other suitable features. One or more controllers including one or more processors and associated memory(s) are disposed within outer housing 110 and function to provide power and control signals to devices connected to console 100; to process user inputs, feedback data, and other data received at console 100; and to control the one or more fluid pumps 140. Suitable hardware and drive mechanisms as part of or in addition to controller may be disposed within outer housing 110 to perform the various functions of console 100 and may include, for example, one or more central processing units (CPU's) and/or microcontroller units (MCU's), power generating and control hardware and corresponding firmware/software stored thereon, sensor circuitry, motors, pump drivers, pump controllers, etc.

The one or more surgical cutting devices 300 may define any suitable configurations for use in performing various different surgical tasks, for use in various different procedures, etc. One example of a suitable surgical cutting device, surgical cutting device 300, generally includes a handle 310, a shaft assembly 320 extending distally from handle 310 (releasably or integrally connected thereto), a cutting tool 330 extending distally from shaft assembly 320 (releasably or integrally connected thereto), a motor 340 disposed within handle 310 and operably coupled to cutting tool 330 to drive rotation and/or reciprocation of cutting tool 330 relative to shaft assembly 320 to cut tissue, and a cord 360 to connect motor 340 to console 100 to enable console 100 to power and control motor 340, thereby controlling cutting tool 330. In aspects, shaft assembly 320 includes a rotation collar 322 that is rotatable relative to handle 310 to advance or retract (depending upon the direction of rotation of rotation collar 322) an outer sleeve 324 of shaft assembly 320 relative to cutting tool 330 to expose more or less of cutting tool 330 at the distal end of outer sleeve 324. Motor 340 may be an electric motor, pneumatic motor, ultrasonic transducer, or other suitable motor configured to drive cutting tool 330 to rotate and/or reciprocate for cutting tissue. Console 100 is configured to drive and control motor 340 such as, for example, a speed, torque, etc. output by motor 340. In aspects, surgical cutting device 300 may include additional features such as, for example, hand control(s), navigation, articulation, etc.

Cutting tool 330 may define any suitable configuration and may be integrated with surgical cutting device 300 or removable therefrom. Various different rotational cutting tools (not explicitly shown) may be configured for releasable attachment with surgical cutting device 300. In aspects, rotational cutting tools are releasably engageable with shaft assembly 320 (which, in turn, may be releasably or integrally connected to handle 310). Alternatively, rotational cutting tools 332 may be integral with corresponding shaft assemblies 320 that are, in turn, releasably engageable with handle 310. In either configuration, surgical cutting device 300 is thus capable of being interchangeably customized with a particular rotational cutting tool, depending upon a particular purpose. Reciprocating cutting tools and/or cutting tools configured for both rotation and reciprocation are also contemplated.

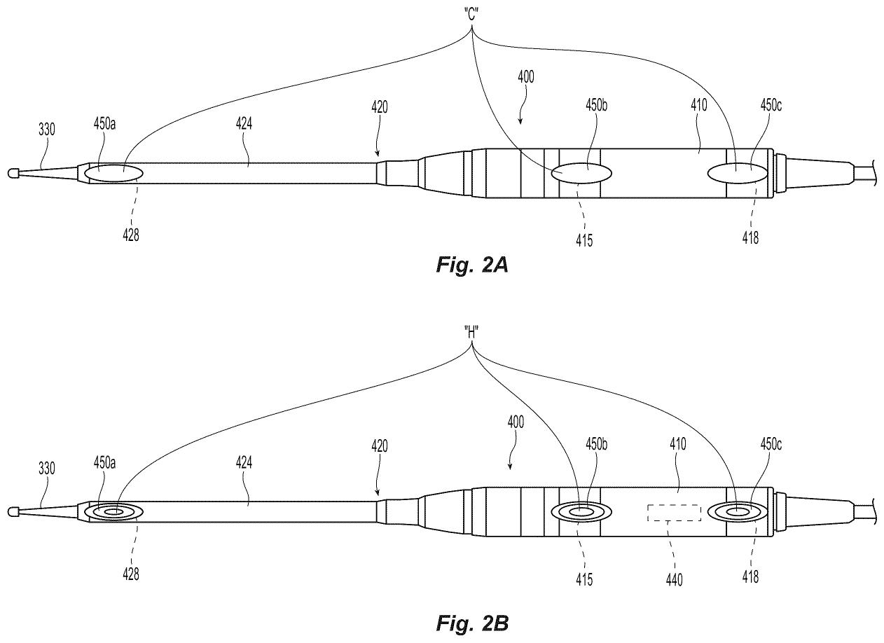

FIGS. 2A and 2B show another embodiment of a surgical cutting device 400 in accordance with the present disclosure including a handle 410, a shaft assembly 420 extending distally from handle 410 (releasably or integrally connected thereto), a cutting tool 430 extending distally from shaft assembly 420 (releasably or integrally connected thereto), a motor 440 disposed within handle 410 and operably coupled to cutting tool 430 to drive rotation and/or reciprocation of cutting tool 430 relative to shaft assembly 420 to cut tissue, and a cord 360 to connect motor 440 to console 100 to enable console 100 to power and control motor 440, thereby controlling cutting tool 430. The details relating to the shaft assembly 420, motor assembly 440 and console 100 are similar to the embodiments described above and, as such, are not described again for the purposes of brevity.

One or more thermochromic indicators 450a-450c are disposed at various locations on the cutting device 400 and are configured to provide a visual indication of a change of condition of one or more internal components of the cutting device 400 based on a change in temperature. A thermochromic indicator is typically made from a thin film of liquid crystal material, inorganic material or organic material that have a wide temperature transition range and that are sensitive to various substances to effect a change. Organic thermochromic indicators are more widely used due to their higher temperature sensitivity, brighter colors, lower cost and non-toxicity.

A first indicator 450a may be placed on the outer surface of shaft 424 proximate an internal bearing 428 near a distal end of shaft 424, a second indicator 450b may be placed on the outer surface of handle 410 proximate an internal bearing 415 within a distal end of handle 410, and a third indicator 450c may be placed on the outer surface of handle 410 proximate an internal bearing 418 near a proximal end of handle 410. In a stable operating environment and under normal operating conditions, the bearings 428, 415, 418 should not produce enough heat to induce a visible change in any one of the indicators 450a-450c, e.g., to cause a thermochromic effect. As a result, all of the indicators 450a-450c should remain in a cool or stable state represented by the reference letter “C” in FIG. 2A. Prior to, during or after use, the surgeon would reference each indicator 450a-450c to determine the state of each respective component 428, 415, and 418. As can be appreciated, additional indicators (not shown) may easily be employed at other locations to monitor other components.

If, for example, during use of one or more of components, e.g., bearings 428, 415, 418, were to generate heat (represented by the letter “H” in FIG. 2B) due to excessive use, wear or some other unstable condition affecting the surgical device 400, this would induce a thermochromic change and the respective indicator 450a-450c would visually change color (or some other visual effect). The indicators 450a-450c may include a wide temperature gradient to provide a greater degree of use or wear of surgical device 400 or two or more indicators may be used on a single component to enable the surgeon to assess a component in varying states of failure, e.g., from “good”, to “fair”, to “poor” and, finally, to “critical”. Again, the surgeon can make this assessment at any time before, during, or after use of the surgical device 400.

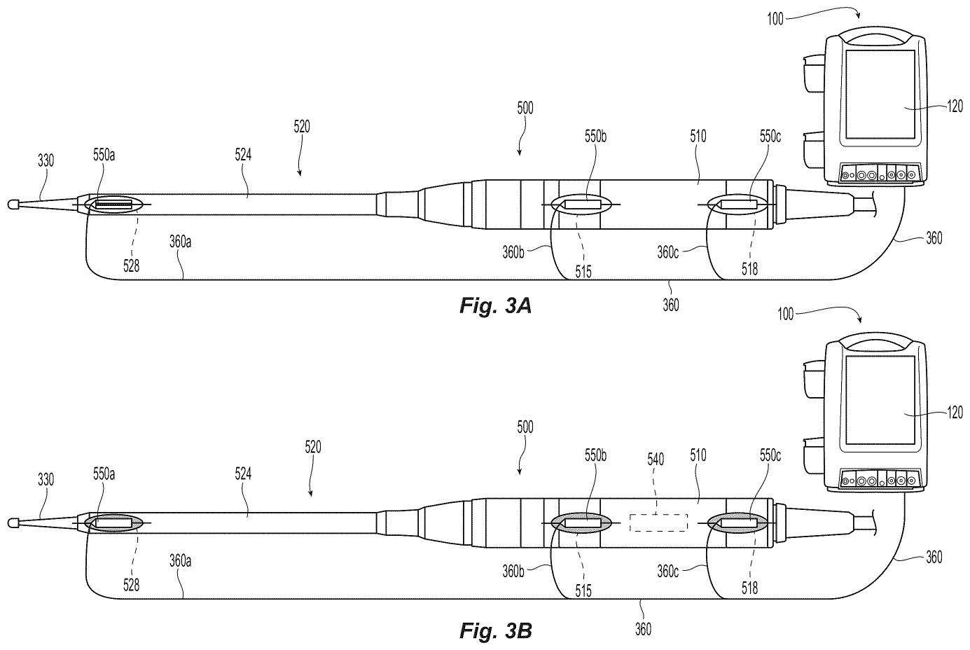

FIGS. 3A and 3B show another embodiment of a surgical cutting device 500 in accordance with the present disclosure including a handle 510, a shaft assembly 520 extending distally from handle 510, a cutting tool 530 extending distally from shaft assembly 520, a motor 540 disposed within handle 510 and operably coupled to cutting tool 530 to drive rotation and/or reciprocation of cutting tool 530 relative to shaft assembly 520 to cut tissue, and a cord 360 to connect motor 540 to console 100 to enable console 100 to power and control motor 540, thereby controlling cutting tool 530. The details relating to the shaft assembly 520, motor assembly 540 and console 100 are similar to the embodiments described above and, as such, are not described again for the purposes of brevity.

One or more thermal-activated elements or fuses 550a-550c are disposed at various locations on the cutting device 500 and are configured to cooperate with the console to provide a visual indication of a change of condition of one or more internal components of the cutting device 500 based on a change in temperature. A thermal-activated element or fuse is a temperature sensing device that acts as a circuit breaker by detecting an overheating condition in an element or circuit and is typically not reusable. Thermal-activated elements or fuses are made from a variety of different materials and in a variety of different ways known in the art.

A first fuse 550a may be placed proximate an internal bearing 528 near a distal end of shaft 524, a second fuse 550b may be placed proximate an internal bearing 515 within a distal end of handle 510, and a third fuse 550c may be placed proximate an internal bearing 518 near a proximal end of handle 510. In a stable operating environment and under normal operating conditions, the bearings 528, 515, 518 should not produce enough heat to trip one of the fuses 550a-550c, e.g., to cause the fuse to short. As a result, all of the fuses 550a-550c should remain in a stable state as shown in FIG. 2A. Prior to, during or after use, the surgeon would reference each fuse 550a-550c to determine the state of each respective component 528, 515, and 518. As can be appreciated, additional fuses (not shown) may easily be employed at other locations to monitor other components.

If, for example, during use one or more of components, e.g., bearings 528, 515, 518, were to generate heat due to excessive use, wear or some other unstable condition affecting the surgical device 500, this would induce one or more fuses to trip, e.g., fuses 550a-550c, which would visually show on GUI 120. Each fuse is independently connected to and monitored by console 100 via a respective lead disposed within cable 360, e.g., fuse 550a connects to console via lead 360a, fuse 550b connects to console via lead 360b, and fuse 550c connects to console via lead 360c. Two or more fuses may be used on a single component to enable the surgeon to assess a component in varying states of failure, e.g., from “good”, to “fair”, to “poor” and, finally, to “critical”. Again, the surgeon can make this assessment at any time before, during, or after use of the surgical device 500.

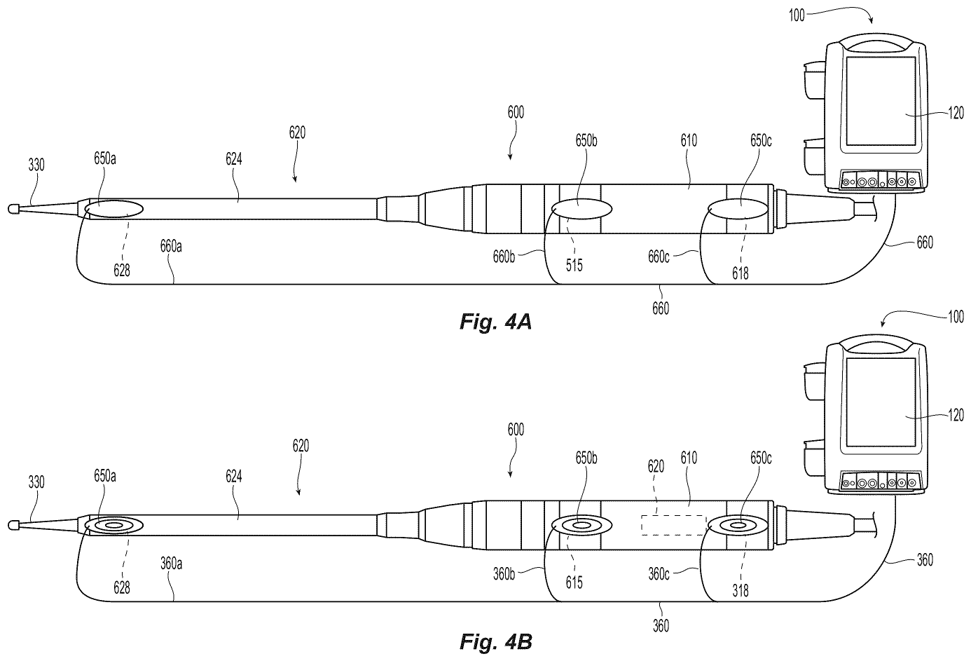

FIGS. 4A and 4B show another embodiment of a surgical cutting device 600 in accordance with the present disclosure including a handle 610, a shaft assembly 620 extending distally from handle 610, a cutting tool 630 extending distally from shaft assembly 620, a motor 640 disposed within handle 610 and operably coupled to cutting tool 630 to drive rotation and/or reciprocation of cutting tool 630 relative to shaft assembly 620 to cut tissue, and a cord 360 to connect motor 640 to console 100 to enable console 100 to power and control motor 640, thereby controlling cutting tool 630. The details relating to the shaft assembly 620, motor assembly 640 and console 100 are similar to the embodiments described above and, as such, are not described again for the purposes of brevity.

One or more thermocouples or thermistors 650a-650c are disposed at various locations on the cutting device 600 and are configured to cooperate with the console 100 to provide a visual indication of a change of condition of one or more internal components of the cutting device 600 based on a change in temperature. A thermocouple is a sensor that measures temperature using two different types of metals joined together at one end. When the junction of the two metals is heated or cooled, a voltage is created that can be correlated back to the temperature.

A first thermocouple 650a may be placed proximate an internal bearing 628 near a distal end of shaft 624, a second thermocouple 650b may be placed proximate an internal bearing 615 within a distal end of handle 610, and a third thermocouple 650c may be placed proximate an internal bearing 618 near a proximal end of handle 610. In a stable operating environment and under normal operating conditions, the bearings 628, 615, 618 should not produce enough heat to cause a voltage spike in thermocouples 650a-650c, e.g., resulting in an alarm condition on console 100. As a result, all of the thermocouples 650a-650c should remain in a stable state as shown in FIG. 2A. Prior to, during or after use, the surgeon would reference each thermocouple 650a-650c to determine the state of each respective component 628, 615, and 618. As can be appreciated, additional thermocouples (not shown) may easily be employed at other locations to monitor other components.

If, for example, during use one or more of components, e.g., bearings 628, 615, 618, were to generate heat due to excessive use, wear or some other unstable condition affecting the surgical device 600, this would cause a voltage spike in one or more of the thermocouples, e.g., fuses 650a-650c, which would visually show on GUI 120. Each thermocouple is independently connected to and monitored by console 100 via a respective lead disposed within cable 360, e.g., thermocouple 650a connects to console via lead 360a, thermocouple 650b connects to console via lead 360b, and thermocouple 650c connects to console via lead 360c. Two or more thermocouples may be used on a single component to enable the surgeon to assess a component in varying states of failure, e.g., from “good”, to “fair”, to “poor” and finally to “critical”. Again, the surgeon can make this assessment at any time before, during, or after use of the surgical device 600.

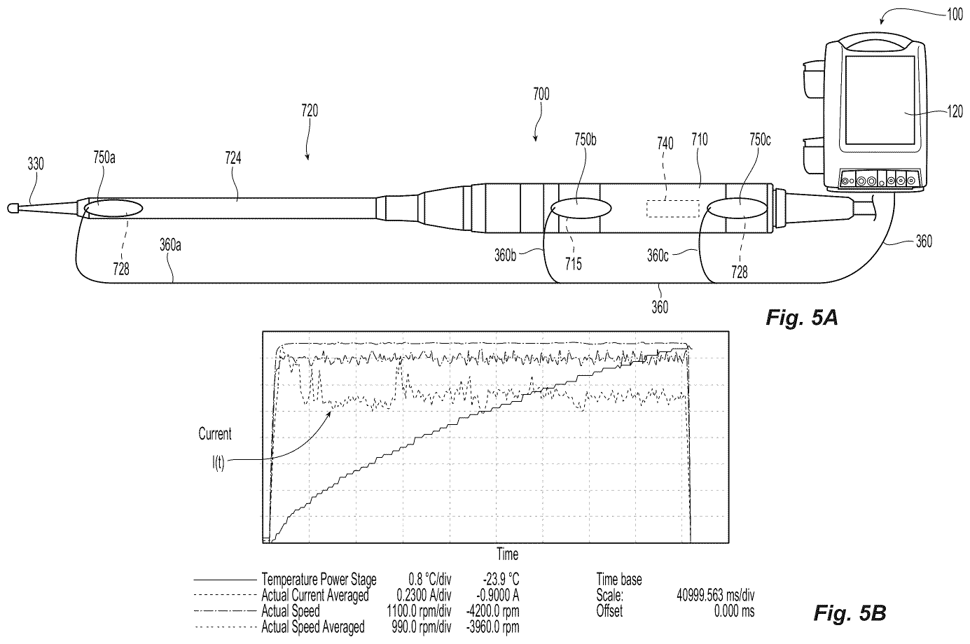

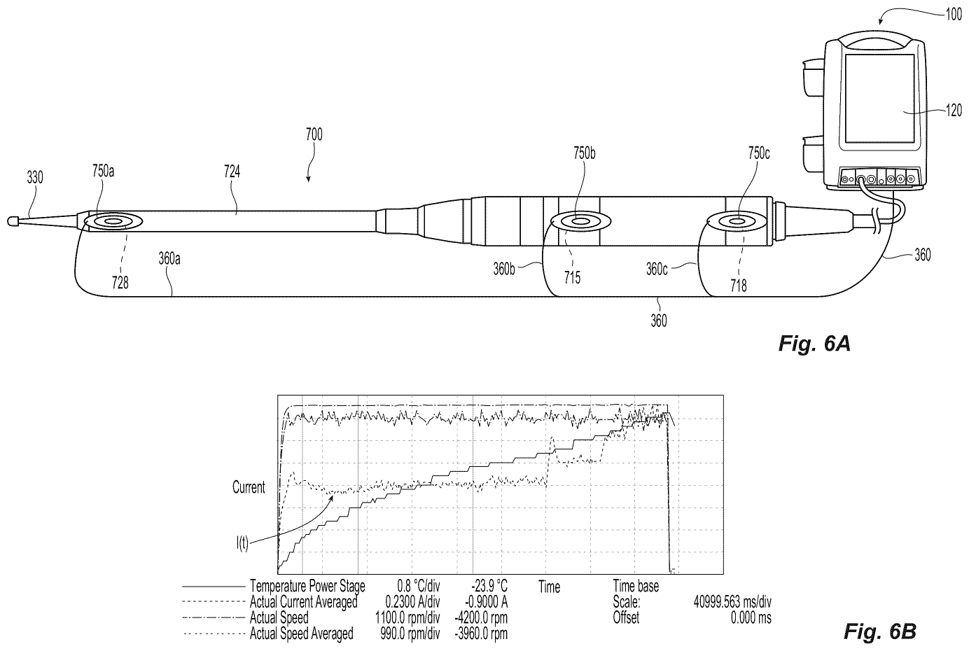

FIGS. 5A-6B show another embodiment of a surgical cutting device 700 in accordance with the present disclosure including a handle 710, a shaft assembly 720 extending distally from handle 710, a cutting tool 730 extending distally from shaft assembly 720, a motor 740 disposed within handle 710 and operably coupled to cutting tool 730 to drive rotation and/or reciprocation of cutting tool 730 relative to shaft assembly 720 to cut tissue, and a cord 360 to connect motor 740 to console 100 to enable console 100 to power and control motor 740, thereby controlling cutting tool 730. The details relating to the shaft assembly 720, motor assembly 740 and console 100 are similar to the embodiments described above and, as such, are not described again for the purposes of brevity.

One or more current contacts 750a-750c are disposed at various locations on the cutting device 700 and are configured to cooperate with the console 100 to provide a visual indication of a change of condition of one or more internal components of the cutting device 700 based on a change in temperature. Each current contact 750a-750c is independently connected to and monitored by console 100 via a respective lead disposed within cable 360, e.g., contact 750a connects to console via lead 360a, contact 750b connects to console via lead 360b, and contact 750c connects to console via lead 360c.

Contact 750a may be placed proximate an internal bearing 728 near a distal end of shaft 724, second contact 750b may be placed proximate an internal bearing 715 within a distal end of handle 710, and third contact 750c may be placed proximate an internal bearing 718 near a proximal end of handle 710. In a stable operating environment and under normal operating conditions, the bearings 728, 715, 718 should not produce enough heat to cause a current demand in a respective contact, e.g., contacts 750a-750c, resulting in an alarm condition on console 100. As a result, all of the contacts 750a-750c should remain in a substantially stable state as shown in the graph FIG. 5B which shows the change in current (I) over time (t) or (I(t)). Prior to, during or after use, the surgeon would reference each contact 750a-750c to determine the state of each respective component 728, 715, and 718. As can be appreciated, additional contacts (not shown) may easily be employed at other locations to monitor other components.

If, for example, during use one or more of components, e.g., bearings 728, 715, 718, were to generate heat due to excessive use, wear or some other unstable condition affecting the surgical device 700, this would cause a spike in current demand over time on one or more of the contacts 750a-750c, which would visually show on GUI 120. Coupling a graphical interface on the GUI 120 (for example as shown in FIG. 6B) would enable the surgeon to assess a component in varying states of failure, e.g., from “good”, to “fair”, to “poor” and, finally, to “critical”. Again, the surgeon can make this assessment at any time before, during, or after use of the surgical device 700.

Alternatively, the console 100 may simply monitor the overall current demand of the surgical device 700 and graph the overall current demand on the GUI 120 as a function of time. If any of the bearings 728, 715, 718, were to generate heat due to excessive use, wear or some other unstable condition affecting the surgical device 700, this would cause a spike in current demand over time in the overall current which would visually show on GUI 120.

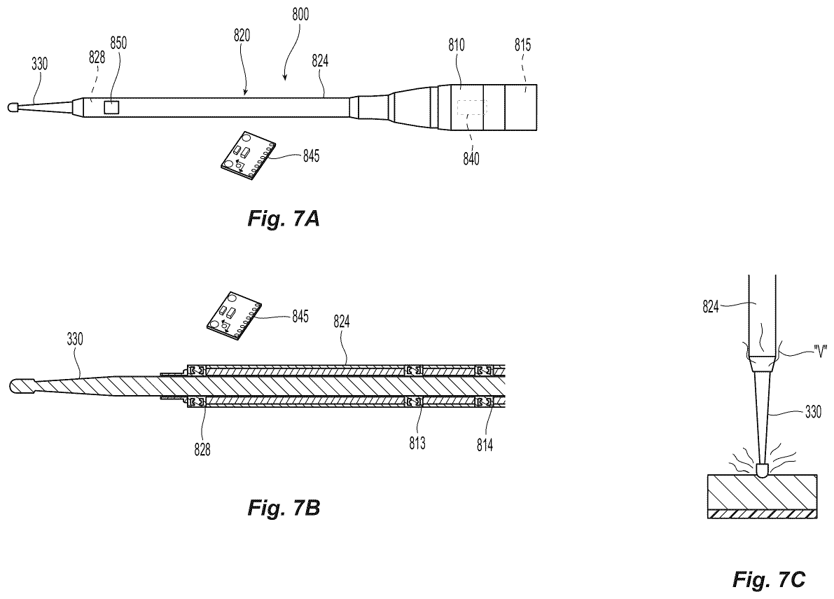

FIGS. 7A-7C show another embodiment of a surgical cutting device 800 in accordance with the present disclosure including a handle 810, a shaft assembly 820 extending distally from handle 810, a cutting tool 830 extending distally from shaft assembly 820, a motor 840 disposed within handle 810 and operably coupled to cutting tool 830 to drive rotation and/or reciprocation of cutting tool 830 relative to shaft assembly 820 to cut tissue, and a cord 360 to connect motor 840 to console 100 to enable console 100 to power and control motor 840, thereby controlling cutting tool 830. The details relating to the shaft assembly 820, motor assembly 840 and console 100 are similar to the embodiments described above and, as such, are not described again for the purposes of brevity.

One or more microcontrollers, e.g., microcontroller 850 (FIG. 7B), may be disposed at various locations on the cutting device 800, e.g., shaft assembly 820 or shaft 824, and are configured to cooperate with the console 100 to actively monitor the various internal components of the cutting device 800 in terms of noise, vibration, current, voltage, etc. During or after use, these parameters are compared against a known neural network of patterns or behaviors to anticipate issues, recommend replacement of components or state of failure of the surgical device 800, e.g., from “good”, to “fair”, to “poor” and finally to “critical”. An accelerometer 845 may be used to monitor the vibration/motion “V” of the various internal components, e.g., bearings 828, 815, and/or 818.

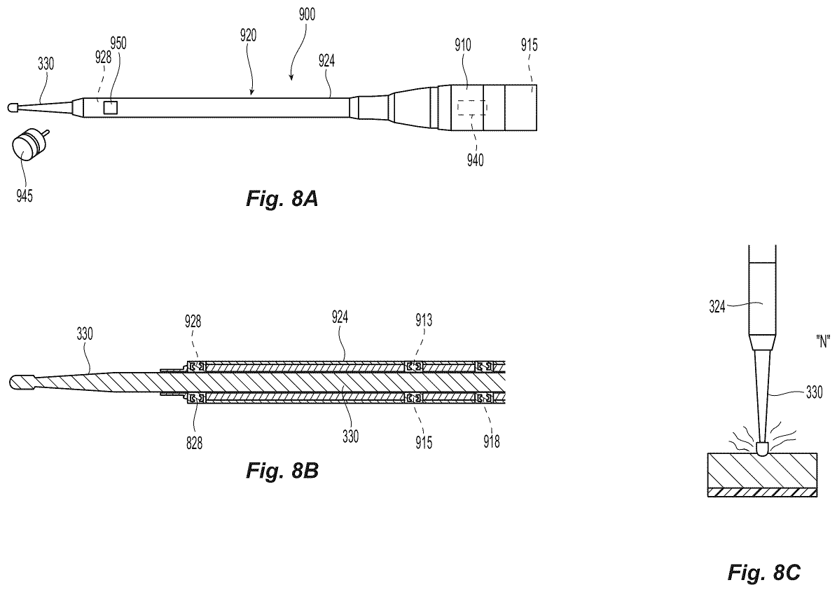

FIGS. 8A-8C show another embodiment of a surgical cutting device 900 in accordance with the present disclosure including a handle 910, a shaft assembly 920 extending distally from handle 910, a cutting tool 930 extending distally from shaft assembly 920, a motor 940 disposed within handle 910 and operably coupled to cutting tool 930 to drive rotation and/or reciprocation of cutting tool 930 relative to shaft assembly 920 to cut tissue, and a cord 360 to connect motor 940 to console 100 to enable console 100 to power and control motor 940, thereby controlling cutting tool 930. The details relating to the shaft assembly 920, motor assembly 940 and console 100 are similar to the embodiments described above and, as such, are not described again for the purposes of brevity.

One or more noise controllers, e.g., noise controller 950 (FIG. 8B), may be disposed at various locations on the cutting device 900 and are configured to cooperate with the console 100 to actively monitor the various internal components of the cutting device 900 in terms of noise, vibration, current, voltage, etc. During or after use, these parameters are compared against a known neural network of patterns or behaviors to anticipate issues, recommend replacement of components or determine a state of failure of the surgical device 900, e.g., from “good”, to “fair”, to “poor” and, finally, to “critical”. Any type of noise sensor, e.g., noise sensor 845, may be used to monitor the noise from the vibration “N” of the various internal components, e.g., bearings 928, 915, and/or 918.

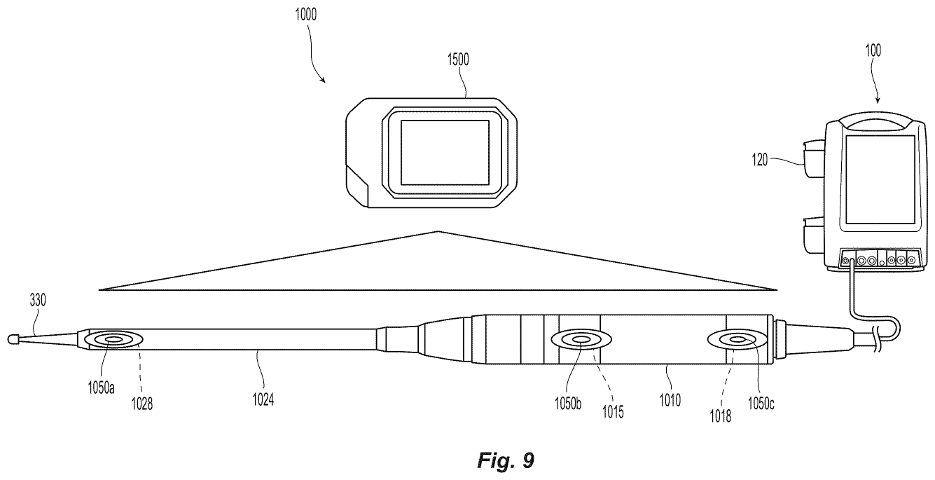

FIG. 9 shows another embodiment of a surgical cutting device 1000 in accordance with the present disclosure including a handle 1010, a shaft assembly 1020 extending distally from handle 1010, a cutting tool 1030 extending distally from shaft assembly 1020, a motor 1040 disposed within handle 1010 and operably coupled to cutting tool 1030 to drive rotation and/or reciprocation of cutting tool 1030 relative to shaft assembly 1020 to cut tissue, and a cord 360 to connect motor 1040 to console 100 to enable console 100 to power and control motor 1040, thereby controlling cutting tool 1030. The details relating to the shaft assembly 1020, motor assembly 1040 and console 100 are similar to the embodiments described above and, as such, are not described again for the purposes of brevity.

A vision-based monitoring system 1500 is disposed at various locations relative to the cutting device 1000 as the cutting device is being used and is configured to provide a visual indication of a change of condition of one or more internal components of the cutting device 1000 based on a change in temperature. Vision-based monitoring systems employing techniques to monitor heat, vibration and cutting performance are envisioned, e.g., using Eulerian video filtering to extrapolate, i.e., anticipate, failures. If there are any subtle changes, e.g., a sudden change in temperature, from normal behavior of the various internal components, e.g., bearings 1028, 1015, 1018, being monitored using the vision-based monitoring system 1500, these changes are then modeled using various filtering techniques such as Eulerian filtering to anticipate if the type or severity of the change would lead to a component failure or the state of failure is identified, e.g., from “good”, to “fair”, to “poor” and, finally, to “critical”.

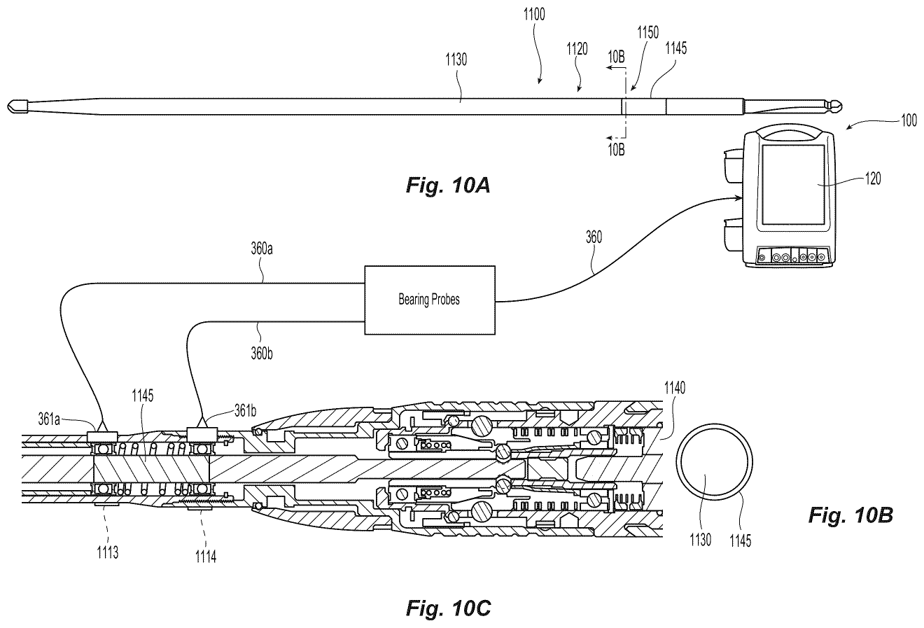

FIGS. 10A-10B show another embodiment of a surgical cutting device 1100 in accordance with the present disclosure including a handle 1110, a shaft assembly 1120 extending distally from handle 1110, a cutting tool 1130 extending distally from shaft assembly 1120, a motor 1140 disposed within handle 1110 and operably coupled to cutting tool 1130 to drive rotation and/or reciprocation of cutting tool 1130 relative to shaft assembly 1120 to cut tissue, and a cord 360 to connect motor 1140 to console 100 to enable console 100 to power and control motor 1140, thereby controlling cutting tool 1130. The details relating to the shaft assembly 1120, motor assembly 1140 and console 100 are similar to the embodiments described above and, as such, are not described again for the purposes of brevity.

The shaft assembly 1120 may include a tool wear indicator 1150 disposed therein configured to communicate with the surgical console 100 to show a current use, or to predict a state of use of, one or more components, e.g., bearings 1113, 1114 (FIG. 10B), prior to, during or after use of the surgical device 1100. More particularly, FIGS. 10A and 10B show the cutting tool 1130 including an electrically conductive paint or coating 1145 applied to a proximal end thereof which is configured to register with bearings 1113, 1114. Each bearing 1113, 1114 is independently connected to and monitored by console 100 via a respective lead disposed within cable 360, e.g., bearing 1113 connects at contact 361a to console via lead 360a, bearing 1114 connects via contact 361b to console 100 via lead 360b.

If, for example, during use the coating 1145 under one of the bearings were to generate heat due to excessive use, wear or some other unstable condition affecting the surgical device 1100, the electrical profile will change proximate the respective bearing, e.g., bearing 1113, which would visually show on GUI 120. The surgeon would be able to assess bearing 1113 in varying states of failure, e.g., from “good”, to “fair”, to “poor” and finally to “critical”. Again, the surgeon can make this assessment at any time before, during, or after use of the surgical device 1100.

While several aspects of the present disclosure have been shown in the drawings, it is not intended that the present disclosure be limited thereto, as it is intended that the present disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. A surgical device, comprising:
a tool;
a motor configured to drive movement of the tool, the tool supported on a shaft assembly, the shaft assembly and the motor including a plurality of components that cooperate to at least one of support or drive the tool; and
at least one indicator disposed proximate at least one of the plurality of components and configured to provide feedback indicative of at least one property of at least one component of the plurality of components either prior to, during or after use of the surgical device,
wherein the plurality of components includes at least one gear, shaft or bearing and the at least one indicator is selected from the group consisting of thermochromic indicators, thermally-activated elements, thermal fuses, thermocouples, and thermistors.

2. The surgical device according to claim 1, wherein the at least one indicator is a thermochromic indicator visibly disposed on an outer surface of the surgical device proximate one or more bearings configured to support the tool, the organic thermochromic indicator having a temperature gradient range to visibly indicate varying states of condition of the one or more bearings of the surgical device.

3. The surgical device according to claim 1, wherein the at least one indicator is a thermally-activated element operably associated with one or more bearings configured to support the tool, the thermally-activated element adapted to couple to an integrated power console to visibly indicate the condition of the one or more bearings of the surgical device.

4. The surgical device according to claim 1, wherein the at least one indicator is a thermocouple operably associated with one or more bearings configured to support the tool, the thermocouple adapted to couple to an integrated power console to visibly indicate varying states of condition of the one or more bearings of the surgical device.

5. The surgical device according to claim 1, wherein the surgical device is adapted to couple to a power console the power console configured to monitor feedback from the at least one indicator during use over time to visibly indicate the condition of the surgical device.

6. The surgical device according to claim 5, wherein the feedback from the at least one indicator is selected from the group consisting of current, voltage, vibration, and noise.

7. A method for determining the state of condition of a surgical device, comprising:
driving a motor to move a tool of a surgical device, the tool supported on a shaft assembly, the shaft assembly and the motor including a plurality of components that cooperate to at least one of support or drive the tool; and
analyzing feedback from at least one indicator disposed proximate at least one of the plurality of components, the feedback indicative of at least one property of the at least one of the plurality of components either prior to, during or after use of the surgical device,
wherein the plurality of components includes at least one gear, shaft or bearing, and the at least one indicator is selected from the group consisting of thermochromic indicators, thermally-activated elements, thermal fuses, thermocouples, and thermistors.

8. The method for determining the state of condition of a surgical device according to claim 7, wherein the analyzing feedback from the at least one indicator includes analyzing feedback from an organic thermochromic indicator visibly disposed on an outer surface of the surgical device proximate one or more bearings configured to support the tool, the thermochromic indicator having a temperature gradient range to visibly indicate varying states of condition of the one or more bearings of the surgical device.

9. The surgical device according to claim 7, wherein the analyzing feedback from the at least one indicator includes analyzing feedback from a thermally-activated element operably associated with one or more bearings configured to support the tool, the thermally-activated element adapted to couple to a power console to visibly indicate the condition of the one or more bearings of the surgical device.

10. The surgical device according to claim 7, wherein the analyzing feedback from the at least one indicator includes analyzing feedback from a thermocouple operably associated with one or more bearings configured to support the tool, the thermocouple adapted to couple to a power console to visibly indicate varying states of condition of the one or more bearings of the surgical device.

11. The surgical device according to claim 7, further comprising: monitoring feedback from the at least one property during use over time to visibly indicate the condition of the surgical device on a power console.

12. The surgical device according to claim 11, wherein the feedback from the at least one indicator is selected from the group consisting of current, voltage, vibration, and noise.

13. A surgical device, comprising:
a tool;
a motor configured to drive movement of the tool, the tool supported on a shaft assembly, the shaft assembly and motor including a plurality of components that cooperate to at least one of support or drive the tool; and
a microcontroller including a sensor operably associated with the shaft assembly and adapted to couple to a power console, the sensor configured to provide feedback indicative of at least one property of the surgical device during use which can be compared against a known neural network of patterns or behaviors to anticipate issues, recommend replacement of one or more of the plurality of components, or determine a state of failure of the surgical device.

14. The surgical device according to claim 13, wherein the sensor is an accelerometer.

15. The surgical device according to claim 13, wherein the sensor is a noise sensor.

16. The surgical device according to claim 13, wherein the feedback from the sensor is selected from the group consisting of drive current, vibration, and noise.

17. The surgical device according to claim 13, wherein the sensor is disposed on a distal end of the shaft assembly.
